(19) United States
(12) Patent Application Publication
(10) Pub. No.: US 2025/0049796 A1
(43) Pub. Date: Feb. 13, 2025

(54) USE OF 4-THIAZOL-N-(PYRIDIN-2-YL)PYRIMIDIN-2-AMINE DERIVATIVES IN COMBINATION THERAPIES FOR CANCER

(71) Applicant: Aucentra Therapeutics Pty Ltd, Adelaide, South Australia (AU)

(72) Inventors: Shudong WANG, Adelaide (AU); Jimma L LENJISA, Woodville North (AU)

(21) Appl. No.: 18/848,000

(22) PCT Filed: Mar. 16, 2023
(86) PCT No.: PCT/AU2023/050183

§ 371 Date: Sep. 17, 2024

(30) Foreign Application Priority Data
Mar. 17, 2022 (AU) 2022900659

(51) Int. Cl. A61K 31/506; A61K 31/5377; A61K 39/395; A61P 35/00

(52) U.S. Cl. CPC A61K 31/506; A61K 31/5377; A61K 39/3955; A61P 35/00

(57) ABSTRACT

A method of treating a proliferative disease or condition in a subject, comprising co-administering to the subject a compound of formula I shown below (or a pharmaceutically acceptable salt, solvate or prodrug thereof): with an immunotherapeutic agent, for example an immune checkpoint inhibitor such as one capable of inhibiting PD-1, PD-L1/2, CTLA-4, BTLA or Tim-3 and/or one or more of the ligands thereof. Pharmaceutical compositions and kits are also disclosed.

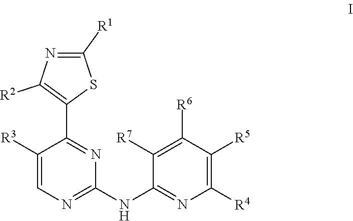

### TECHNICAL FIELD

The present disclosure relates to combination therapies for treating proliferative cell diseases and conditions. In one particular application, the disclosure provides a method which comprises administering to a subject such as a cancer patient, an effective amount of a particular thiazole-pyrimidine anti-proliferative compound(s) in combination with an immune checkpoint inhibitor(s) such as an anti-PD-1 antibody.

### PRIORITY DOCUMENT

The present application claims priority from Australian Provisional Patent Application No. 2022900659 titled “USE OF 4-THIAZOL-N—(PYRIDIN-2-YL)PYRIMIDIN-2-AMINE DERIVATIVES IN COMBINATION THERAPIES FOR CANCER” and filed on 17-3-2022, the content of which is hereby incorporated by reference in its entirety.

### BACKGROUND

Methods of cancer immunotherapy that enhance a patient's immune system to target tumours have recently been developed (see, for example, Rizvi N A et al., Science 348:124-8, 2015; and Pardoll D M., Nat Rev Cancer 12:252-64, 2012). One class of such methods has arisen through the discovery of immune checkpoint receptors, such as PD-1 (programmed cell death protein 1; CD279) and CTLA-4 (cytotoxic T-lymphocyte-associated protein 4), that repress the activity of anti-tumour (cytotoxic) T cells. In particular, this discovery has led to the development of blocking antibodies directed against certain examples of these receptors (or their ligands), including for example, pembrolizumab (a humanised antibody directed against PD-1), nivolumab (another anti-PD-1 antibody), atezolizumab (a monoclonal antibody directed against the PD-1 ligand, PD-L1, so as to inhibit interaction between PD-1 and PD-L1), durvalumab (another anti-PD-L1 antibody) and ipilimumab (a monoclonal antibody directed against CTLA-4). Some patients treated with checkpoint inhibitors such as these experience a durable tumour regression (Sharma P et al., Cell 161:205-14, 2015). However, despite promising clinical results, therapies using checkpoint inhibitor(s) (ie termed “checkpoint inhibitor therapy” or “checkpoint blockade therapy”) have, to-date, only proven to be successful in subset(s) of patients (Havel J J et al., Nat Rev Cancer 19(3):133-150, 2019). Moreover, increasingly, evidence is emerging which shows that tumours can acquire resistance against immunotherapies such as checkpoint inhibitor therapy (Koyama S, et al. Nat Commun 7:10501, 2016). Thus, it is desirable to develop new treatment strategies that may broaden the range of tumours and/or patients that respond to immunotherapy, or which may otherwise enhance the specificity and/or efficacy of existing immunotherapies such as those mentioned above.

One potential new strategy is to identify and develop small molecule kinase inhibitors which might enhance or complement an immunotherapy. Small molecule inhibitors have many advantages in comparison with antibody-based biologics including, for example, greater exposure in the tumour microenvironment and access to intracellular targets (Petroni G et al., Nat Review Immunol 20:669-679, 2020). Moreover, there is some evidence to indicate that signalling from oncogenic kinase drivers can alter the tumour microenvironment to promote immunosuppression (Ahn R and J Ursini-Siegel, Int J Mol Sci 22(5):2608-2631, 2021), and the inhibition of these oncogenes using small molecule kinase inhibitors can potentially contribute to immune reactivation. In addition, small molecule kinase inhibitors can directly alter immune cell function and contribute to antitumour immunity (Ahn and Ursini-Siegel, 2021 supra). As such, combining small molecule kinase inhibitors, which can induce dramatic but short-lived tumour regression, with an immunotherapy such as checkpoint inhibitor therapy, which provide slower but potentially more durable responses, is an attractive potential new treatment strategy.

The present Applicant has identified and developed a range of novel small molecule kinase inhibitors, particularly directed to cyclin-dependent kinases (CDKs). CDKs are known to be associated with various cyclin subunits, playing pivotal roles in the regulation of a variety of important regulatory pathways in cells, including cell-cycle control, apoptosis, neuronal physiology, differentiation and transcription. As such, aberrant CDK expression and/or activity can cause or contribute to, inter alia, aberrant cell-cycle control, resulting in unlimited cell cycle re-entry and progression, which is a hallmark of human cancers. To date, at least 20 CDKs and 30 cyclins have been identified. They may be classified into two major groups, reflecting their functions, the cell cycle regulator CDKs and the transcription regulator CDKs (Wang S et al., Trends Pharmacol Sci 29(6):302-313, 2008; Diab S et al., J Med Chem 63(14):7458-7474, 2020). The class of the cell cycle regulator CDKs includes CDK 1, 2, 3, 4, 5, 6 and 7 and these function with their cyclin partners (eg cyclins A, B, C, D1, D2, D3, E and F) to regulate promotion of the cell cycle. The class of the transcription regulator CDKs includes CDK 7, 8, 9 and 11, which work together with cyclins C, H, K, L1, L2, Ti and T2. Given the functions of the CDK classes, it is not surprising that CDKs have been implicated in cell proliferation diseases and conditions, particularly cancer. Cell proliferation is a result of the direct or indirect deregulation of the cell division cycle and the CDKs play a critical role in the regulation of the various phases of this cycle. Therefore, CDK inhibitors are useful targets for cancer therapy, and CDK4/6 inhibitors such as palbociclib, ribociclib and abemaciclib have received US Food and Drug Administration (FDA) approval for the treatment of patients with advanced or metastatic breast cancers as a single agent or in combination. Although best known for their function in the blockade of cell-cycle progression, accumulating evidence indicates that the anti-cancer effects of CDK4/6 inhibitors such as these also results from a range of immunostimulatory effects as they interact with malignant cells as well as with immune cell populations of the tumour microenvironment (Petroni G et al., 2020 supra).

The present Applicant has identified a group of thiazole-pyrimidine CDK inhibitor compounds that show significant potential to enhance the specificity and/or efficacy of various agents for cancer immunotherapy such as those used in checkpoint inhibitor therapy (eg an anti-PD-1 blocking antibody), and might therefore form the basis of useful novel combination therapies.

### SUMMARY

In a first aspect, the present disclosure provides a method of treating a proliferative disease or condition in a subject, comprising co-administering to the subject a compound of formula I shown below (or a pharmaceutically acceptable salt, solvate or prodrug thereof) with an immunotherapeutic agent:

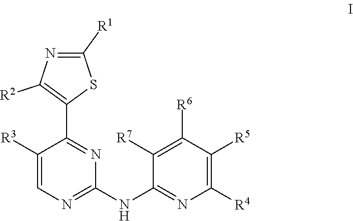

wherein:
R¹, R², R³, R⁴, R⁵, R⁶ and R⁷ are each independently selected from the group consisting of H, alkyl, aryl, aralkyl, halogen, NO₂, CN, CF₃, OH, O-alkyl, O-aryl, NH₂, NH-alkyl, NH-aryl, N-(alkyl)₂, N-(aryl)₂, N-(alkyl)(aryl), COOH, CONH₂, CONH-alkyl, CONH-aryl, SO₃H, SO₂-alkyl, SO₂-aryl, SO₂NH₂, CF₃, CO-alkyl, CO-aryl, wherein said alkyl, aryl and aralkyl groups may be optionally substituted with one or more groups selected from halogen, CN, OH, O-methyl, NH₂, COOH, CONH₂ and CF₃, and heterocyclic groups optionally substituted with one or more groups selected from alkyl, NH₂, NH-alkyl, N(alkyl)₂, COH and CO-alkyl;
but wherein the compound is not 5-(2-((5-(4-(dimethylamino)piperidin-1-yl)pyridin-2-yl)amino)-5-fluoropyrimidin-4-yl)-N,4-dimethylthiazol-2-amine.

The immunotherapeutic agent may be selected from, for example, agents that are known to those skilled in the art as being capable of promoting an immunoreaction against cancer cells, such as an immune checkpoint inhibitor.

In a second aspect, the present disclosure provides a pharmaceutical composition for treating a proliferative disease or condition in a subject, comprising a compound of formula I as defined in the first aspect (or a pharmaceutically acceptable salt, solvate or prodrug thereof) and an immunotherapeutic agent.

In a third aspect, the present disclosure provides the use of a compound of formula I as defined in the first aspect (or a pharmaceutically acceptable salt, solvate or prodrug thereof) and an immunotherapeutic agent for treating a proliferative disease or condition in a subject.

In a fourth aspect, the present disclosure provides the use of a compound of formula I as defined in the first aspect (or a pharmaceutically acceptable salt, solvate or prodrug thereof) and an immunotherapeutic agent in the manufacture of a medicament for treating a proliferative disease or condition in a subject.

In a fifth aspect, the present disclosure provides a kit comprising first and second containers (eg vials), wherein the first container contains a compound of formula I as defined in the first aspect (or a pharmaceutically acceptable salt, solvate or prodrug thereof), and the second container contains an immunotherapeutic agent; optionally packaged with instructions for the use of the kit in the method of the first aspect.

### BRIEF DESCRIPTION OF FIGURES

FIG. 1 provides graphical results demonstrating the in vivo anti-tumour activity of compound 1 (N-cyclopentyl-5-(2-((5-((4-ethylpiperazin-1-yl)methyl)pyridin-2-yl)amino)-5-fluoropyrimidin-4-yl)-4-methylthiazol-2-amine) against syngeneic EMT6 triple-negative breast cancer cells in mice (A). The treatment reduced the level of RB phosphorylation (B) and the immune checkpoint protein ligand PD-L1 (C) in tumourous tissue, indicating that the compound provides CDK4/6 inhibition and immunotherapeutic effects, respectively;

FIG. 2 provides further graphical results demonstrating the in vivo anti-tumour activity of compound 1 against CT26.WT syngeneic colorectal cancer cells in mice. The animals were treated with vehicle or compound 1 alone (A), or compound 1 in combination with an anti-PD1 antibody (B). The anti-tumour efficacy translated to the extended survival of animals (C); and

FIG. 3 provides yet further graphical results showing the in vivo anti-tumour efficacy of compound 1 in the CT26.WT syngeneic mouse model through activation of immunity. The animals were treated with vehicle or compound 1 (A), an anti-PD1 antibody (B), or compound 1 in combination with anti-PD1 antibody (C). The increased T cell population in the spleen of the treated mice was detected (D-G).

### DETAILED DESCRIPTION

The present Applicant has identified a group of 4-thiazol-N-(pyridin-2-yl)pyrimidin-2-amine derivatives suitable for enhancing the activity and/or efficacy of a cancer immunotherapy (eg a therapy involving the administration of an immunotherapeutic agent that promotes an immunoreaction against cancer cells such as those used in checkpoint inhibitor therapy (eg an anti-PD-1 antibody)). As shown in Example 2, it was found that a representative compound, (N-cyclopentyl-5-(2-((5-((4-ethylpiperazin-1-yl)methyl)pyridin-2-yl)amino)-5-fluoropyrimidin-4-yl)-4-methylthiazol-2-amine) (“compound 1”), is capable of an anti-proliferative effect through CDK4/6 inhibition and associated immunotherapeutic mode(s) of action (eg the modulation of regulatory T cells, and/or PD-1/PD-1 ligands).

In a first aspect, the present disclosure provides a method of treating a proliferative disease or condition in a subject, comprising co-administering to the subject a compound of formula I shown below (or a pharmaceutically acceptable salt, solvate or prodrug thereof) with an immunotherapeutic agent:

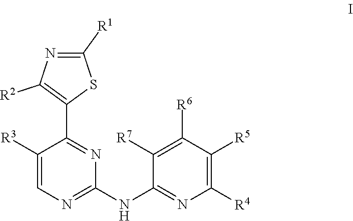

wherein:
R¹, R², R³, R⁴, R⁵, R⁶ and R⁷ are each independently selected from the group consisting of H, alkyl, aryl, aralkyl, halogen, NO₂, CN, CF₃, OH, O-alkyl, O-aryl, NH₂, NH-alkyl, NH-aryl, N-(alkyl)₂, N-(aryl)₂, N-(alkyl)(aryl), COOH, CONH₂, CONH-alkyl, CONH-aryl, SO₃H, SO₂-alkyl, SO₂-aryl, SO₂NH₂, CF₃, CO-alkyl, CO-aryl, wherein said alkyl, aryl and aralkyl groups may be optionally substituted with one or more groups selected from halogen, CN, OH, O-methyl, NH₂, COOH, CONH₂ and CF₃, and heterocyclic groups optionally substituted with one or more groups selected from alkyl, NH₂, NH-alkyl, N(alkyl)₂, COH and CO-alkyl;
but wherein the compound is not 5-(2-((5-(4-(dimethylamino)piperidin-1-yl)pyridin-2-yl)amino)-5-fluoropyrimidin-4-yl)-N,4-dimethylthiazol-2-amine.

The compounds of formula I have been found to possess anti-proliferative activity (eg anti-cancer effects); it is considered that these compounds block tumour cell proliferation by inhibiting the activity of CDK4 and CDK6). In particular, compounds of formula I have been found to, for example, inhibit cell growth of cancer cell lines both in vitro and in vivo and are considered to be useful on their own in the treatment of proliferative cell diseases and conditions.

Further, the compounds of formula I have been found to possess anti-proliferative activity (eg anti-cancer effects) through previously unrecognised immunotherapeutic effects. In particular, the compounds of formula I may, for example, inhibit of cancer cell growth in vivo through promoting an immunoreaction against the cancer cells.

As such, it is considered that the compounds of formula I have excellent potential to additionally act as an immunotherapeutic agent, especially when used in combination with another immunotherapeutic agent such as those used in checkpoint inhibitor therapy (eg an anti-PD-1 blocking antibody), for the treatment of proliferative cell diseases and conditions.

In particular, as shown in the Examples hereinafter, when mice models bearing syngeneic allografts of cancer cell lines were co-administered with a compound of formula I and another immunotherapeutic agent (in particular, an immune checkpoint inhibitor), enhanced anti-proliferative effects and increased survival rates were achieved (ie relative to the use of the formula I compound or other immunotherapeutic agent alone).

In preferred embodiments, the enhanced anti-proliferative effects represents synergism between the formula I compound and the other immunotherapeutic agent, as may be assessed by, for example, determining combination index (CI) values according to any of the methodologies well known to those skilled in the art, including the Chou-Talaly method (Chou T C et al., Trends Pharmacol Sci 4:450-454, 1983), wherein CI values of <1 indicate a synergistic interaction between the formula I compound and the other immunotherapeutic agent. In some embodiments, the CI values are determined on the basis of the reduced level of tumour volume achieved by the combination treatment in an in vivo mouse model such as has been described in the Examples hereinafter.

In this specification, a number of terms and phrases are used which are well known to those skilled in the art. Nevertheless, for the purposes of clarity, a number of these terms and phrases are hereinafter defined.

As used herein, an anti-proliferative activity or effect within the scope of the present disclosure may be demonstrated by the ability to inhibit cell proliferation in an in vitro whole cell assay and/or the ability to reduce tumour volume in vivo. An example(s) of suitable assays for such activity, including methods for performance, are described in the Examples hereinafter.

The term “treating”, as used herein, includes prophylaxis as well as the alleviation of established symptoms of a disease or condition. As such, the act of “treating” a disease or condition therefore includes: (1) preventing or delaying the appearance of clinical symptoms of the disease or condition developing in a subject afflicted with or predisposed to the disease or condition; (2) inhibiting the disease or condition (ie arresting, reducing or delaying the development of the disease or condition or a relapse thereof (in case of a maintenance treatment)) or at least one clinical or subclinical symptom thereof; and (3) relieving or attenuating the disease or condition (ie causing regression of the disease or condition or at least one of its clinical or subclinical symptoms).

As used herein, the term “alkyl” includes straight chain alkyl groups, branched alkyl groups and cyclic alkyl groups having from 1 to 8 carbon atoms (eg methyl, ethyl propyl, isopropyl, butyl, isobutyl, tert-butyl, pentyl, hexyl, cyclopropyl, cyclobutyl, cyclopentyl etc).

As used herein, the term “aryl” refers to a substituted (mono- or poly-) or unsubstituted monoaromatic or polyaromatic group, wherein said polyaromatic group may be fused or unfused. The term therefore includes groups having from 6 to 10 carbon atoms (eg phenyl, naphthyl etc). It is also to be understood that the term “aryl” is synonymous with the term “aromatic”.

As used herein, the term “aralkyl” is used as a conjunction of the terms alkyl and aryl as defined above.

The term “aliphatic” takes its normal meaning in the art and includes non-aromatic groups such as alkanes, alkenes and alkynes and substituted derivatives thereof.

As used herein, the term “alicyclic” refers to a cyclic aliphatic group.

The term “halogen” refers to fluoro, chloro, bromo and iodo.

As used herein, the term “heterocyclic” refers to a saturated or unsaturated cyclic group comprising one or more heteroatoms (eg N) in a ring system (eg a system comprising one or more rings (mono- or poly-), and wherein where more than one ring is present, the rings may be fused and/or unfused). As such, the term covers saturated heterocyclic groups such as a pyrrolidinyl, morpholinyl, aziridine and piperazine, and unsaturated heterocyclic groups (“heteroaryl” groups such as 2-pyridyl, 3-pyridyl, 4-pyridyl, 4-pyrimidyl, 5-indolyl, furan, thiophene and thiazole etc); and wherein at least one ring of the ring system contains from one to four heteroatoms selected from N, O and S as ring members (ie it contains at least one heterocyclic ring), and wherein the nitrogen and sulfur atoms can be oxidised and the nitrogen atom(s) can be quaternised. A heterocyclic group can be attached to the remainder of the molecule through an annular carbon or annular heteroatom, and it can be attached through any ring of the ring system, if that ring system is, for example a poly-ring system such as a bicyclic, tricyclic or fused ring system.

The term “derivative” as used herein, includes any chemical modification of an entity. Illustrative of such chemical modifications is the replacement of hydrogen by a halogen group, an alkyl group, an acyl group or an amino group.

As used herein, the phrase “manufacture of a medicament” includes the use of a compound of formula I and/or the immunotherapeutic agent directly as the medicament or in any stage of the manufacture of a medicament comprising he compound of formula I and/or the immunotherapeutic agent.

Some of the compounds of formula I may exist as single stereoisomers, racemates, and/or mixtures of enantiomers and/or diastereomers. All such single stereoisomers, racemates and mixtures thereof, are encompassed within the scope of the present disclosure. The isomeric forms such as diastereomers, enantiomers, and geometrical isomers can be separated by physical and/or chemical methods known to those skilled in the art.

The term “pharmaceutically acceptable salt” as used herein, refers to salts that retain the desired biological activity of the compounds of formula I, and include pharmaceutically acceptable acid addition salts and base addition salts. Suitable pharmaceutically acceptable acid addition salts of the compounds of formula I may be prepared from an inorganic acid or from an organic acid. Examples of such inorganic acids are hydrochloric, sulfuric and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic. Additional information on pharmaceutically acceptable salts can be found in Remington's Pharmaceutical Sciences, 19th Edition, Mack Publishing Co, Easton PA 1995.

The term “solvate” refers to any form of the compound of formula I, resulting from solvation of with an appropriate solvent. Such a form may be, for example, a crystalline solvate or a complex that may be formed between the solvent and the dissolved compound.

The term “prodrug” means a compound that undergoes conversion to a compound of formula I within a biological system, usually by metabolic means (eg by hydrolysis, reduction or oxidation). For example, an ester prodrug of a compound of formula I containing a hydroxyl group may be convertible by hydrolysis in vivo to the compound of formula I. Suitable esters of the compounds of formula I containing a hydroxyl group may be, for example, acetates, citrates, lactates, tartrates, malonates, oxalates, salicylates, propionates, succinates, fumarates, maleates, methylene-bis-P-hydroxynaphthoates, gestisates, isethionates, di-p-toluoyltartrates, methanesulfonates, ethanesulfonates, benzenesulfonates, p-toluenesulfonates, cyclohexylsulfamates and quinates. As another example, an ester prodrug of a compound of formula I containing a carboxy group may be convertible by hydrolysis in vivo to the compound of formula I. Examples of ester prodrugs include those described by Leinweber F J, Drug Metab Rev 18:379-439 (1987). Similarly, an acyl prodrug of a compound of formula I containing an amino group may be convertible by hydrolysis in vivo to the compound of formula I. Examples of prodrugs for these and other functional groups, including amines, are provided in Prodrugs: challenges and rewards, Valentino J Stella (ed), Springer, 2007.

In the case of compounds of formula I that are solid, it will be understood by those skilled in the art that the compounds (or pharmaceutically acceptable salts, solvates or prodrugs thereof) may exist in different crystalline or polymorphic forms, all of which are encompassed within the scope of the present disclosure.

The term “therapeutically effective amount” or “effective amount” is an amount sufficient to effect beneficial or desired clinical results. A therapeutically effective amount can be administered in one or more administrations. Typically, a therapeutically effective amount is sufficient for treating a disease or condition or otherwise to palliate, ameliorate, stabilise, reverse, slow or delay the progression of a disease or condition such as, for example, cancer or another proliferative cell disease or condition. By way of example only, a therapeutically effective amount of a compound of formula I, or a pharmaceutically acceptable salt, solvate or prodrug thereof, may comprise between about 0.1 and about 250 mg/kg body weight per day, more preferably between about 0.1 and about 100 mg/kg body weight per day and, still more preferably between about 0.1 and about 25 mg/kg body weight per day. For the other immunotherapeutic agent, a therapeutically effective amount may be expected to vary considerably depending upon the particular immunotherapeutic agent, but may similarly comprise between about 0.1 and about 250 mg/kg body weight every 1-3 weeks, more preferably between about 0.1 and about 100 mg/kg body weight every 1-3 weeks and, still more preferably between about 0.1 and about 25 mg/kg body weight every 1-3 weeks. However, notwithstanding the above, it will be understood by those skilled in the art that the therapeutically effective amount of the compound/agent may also vary and depend upon a variety of factors including the activity of the particular compound/agent, the metabolic stability and length of action of the particular compound/agent, the age, body weight, sex, health, route and time of administration, rate of excretion of the particular compound/agent, and the severity of, for example, the cancer or other proliferative cell disease or condition to be treated.

In some embodiments, R¹ is H, alkyl (eg a C₁₋₆ alkyl or, preferably, a C₁₋₃ alkyl such as methyl, ethyl and C(CH₃)₂ or a C₃₋₆ cycloalkyl such as cyclopentyl), or NH—C₁₋₆ alkyl (eg an NH— C₁₋₃ alkyl such as NH-methyl and NH-ethyl, or an NH— C₃₋₆ cycloalkyl such as NH-cyclopentyl). Most preferably, R¹ is NH—C₁₋₆ alkyl (eg an NH—C₁₋₃ alkyl such as NH-methyl and NH-ethyl, or an NH—C₃₋₆ cycloalkyl such as NH-cyclopentyl).

In some embodiments, R² is H, alkyl (eg a C₁₋₆ alkyl or, preferably, a C₁₋₃ alkyl such as methyl or ethyl), CN or halogen (preferably F).

In some embodiments, R³ is H, alkyl (eg a C₁₋₆ alkyl), CN or halogen (preferably F).

In some embodiments, one or both of R⁴ and R⁷ is/are (independently) H, O—C₁₋₆ alkyl or halogen.

In some embodiments, one or both of R⁵ and R⁶ is/are (independently) a heterocyclic group (preferably a saturated or unsaturated 5- or 6-membered cyclic group comprising one or two N heteroatoms) optionally substituted with one or more groups selected from alkyl (eg a C₁₋₆ alkyl or, preferably, a C₁₋₃ alkyl such as methyl, ethyl or C(CH₃)₂), NH₂, NH-alkyl such as NH-methyl and NH-ethyl, N(alkyl)₂ such as N(C₁₋₃ alkyl)₂ (eg N(CH₃)₂, N(CH₂CH₃)₂ and N(CH₃)(CH₂CH₃)), COH and CO—(C₁₋₃ alkyl) (eg COCH₃). The heterocyclic group(s) may be provided with an alkyl bridge (eg a —CH₂— or —CH₂CH₂— bridge) linking the group to the carbon atom at position 4/5 of the pyridine ring.

Most preferably, R⁵ and R⁶ is/are independently selected from the following:

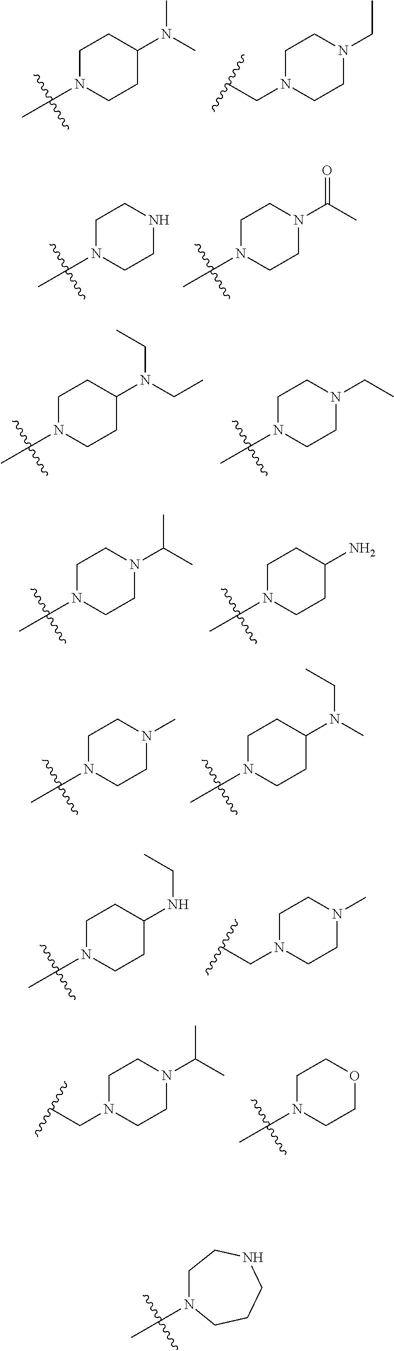

In some embodiments, R⁴, R⁶ and R⁷ are all H.

In one especially preferred embodiment, the compound of formula I is:
N-cyclopentyl-5-(2-((5-((4-ethylpiperazin-1-yl)methyl)pyridin-2-yl)amino)-5-fluoropyrimidin-4-yl)-4-methylthiazol-2-amine;
N-cyclopentyl-4-methyl-5-(2-((5-(piperazin-1-yl)pyridin-2-yl)amino)pyrimidin-4-yl)thiazol-2-amine;
N-cyclopentyl-5-(2-((5-(4-ethylpiperazin-1-yl)pyridin-2-yl)amino)pyrimidin-4-yl)-4-methylthiazol-2-amine;
2-((5-(4-acetylpiperazin-1-yl)pyridin-2-yl)amino)-4-(4-methyl-2-(methylamino)thiazol-5-yl)pyrimidine-5-carbonitrile;
N-cyclopentyl-5-(2-((5-((4-ethylpiperazin-1-yl)methyl)pyridin-2-yl)amino)pyrimidin-4-yl)-4-methylthiazol-2-amine;
5-(2-((5-(4-aminopiperidin-1-yl)pyridin-2-yl)amino)-5-fluoropyrimidin-4-yl)-N,4-dimethylthiazol-2-amine;
5-(2-((5-(4-aminopiperidin-1-yl)pyridin-2-yl)amino)pyrimidin-4-yl)-N-cyclopentyl-4-methylthiazol-2-amine;
N-cyclopentyl-5-(5-fluoro-2-((5-morpholinopyridin-2-yl)amino)pyrimidin-4-yl)-4-methylthiazol-2-amine;
5-(2-((5-(4-(ethylamino)piperidin-1-yl)pyridin-2-yl)amino)-5-fluoropyrimidin-4-yl)-N,4-dimethylthiazol-2-amine;
5-(2-((5-(4-(ethyl(methyl)amino)piperidin-1-yl)pyridin-2-yl)amino)-5-fluoropyrimidin-4-yl)-N,4-dimethylthiazol-2-amine;
5-(5-fluoro-2-((5-((4-methylpiperazin-1-yl)methyl)pyridin-2-yl)amino)pyrimidin-4-yl)-N,4-dimethylthiazol-2-amine;
5-(5-fluoro-2-((5-((4-isopropylpiperazin-1-yl)methyl)pyridin-2-yl)amino)pyrimidin-4-yl)-N,4-dimethylthiazol-2-amine; or
5-(2-((5-(4-(diethylamino)piperidin-1-yl)pyridin-2-yl)amino)-5-fluoropyrimidin-4-yl)-N,4-dimethylthiazol-2-amine.

In another especially preferred embodiment, the compound of formula I is:
N-cyclopentyl-5-(2-((5-((4-ethylpiperazin-1-yl)methyl)pyridin-2-yl)amino)-5-fluoropyrimidin-4-yl)-4-methylthiazol-2-amine;
N-cyclopentyl-4-methyl-5-(2-((5-(piperazin-1-yl)pyridin-2-yl)amino)pyrimidin-4-yl)thiazol-2-amine;
N-cyclopentyl-5-(2-((5-(4-ethylpiperazin-1-yl)pyridin-2-yl)amino)pyrimidin-4-yl)-4-methylthiazol-2-amine;
2-((5-(4-acetylpiperazin-1-yl)pyridin-2-yl)amino)-4-(4-methyl-2-(methylamino)thiazol-5-yl)pyrimidine-5-carbonitrile; or
N-cyclopentyl-5-(2-((5-((4-ethylpiperazin-1-yl)methyl)pyridin-2-yl)amino)pyrimidin-4-yl)-4-methylthiazol-2-amine.

In yet another preferred embodiment, the compound of formula I is N-cyclopentyl-5-(2-((5-((4-ethylpiperazin-1-yl)methyl)pyridin-2-yl)amino)-5-fluoropyrimidin-4-yl)-4-methylthiazol-2-amine.

In some preferred embodiments, the compound of formula I exhibits anti-proliferative activity in human cell lines, as measured by a standard cytotoxicity assay. Preferably, the compound exhibits an IC₅₀ value of less than 5 μM, even more preferably less than 1 μM as measured by a standard cell viability assay. More preferably still, the compound exhibits an IC₅₀ value of less than 0.5 μM.

In some preferred embodiments, the compound of formula I inhibits CDK4 and/or CDK6, as measured by any standard assay well known to those skilled in the art. Preferably, the compound exhibits an IC₅₀ value of less than 1 μM or less than 0.5 μM as measured by any standard kinase assay well known to those skilled in the art, more preferably still less than 0.1 μM.

Particular examples of compounds of formula I for use in the method of the first aspect are shown in Table 1 below.

TABLE 1
Chemical structure of selected compounds of the present disclosure

| No. | Structure | Name | Mass |
|---|---|---|---|
| 1 | 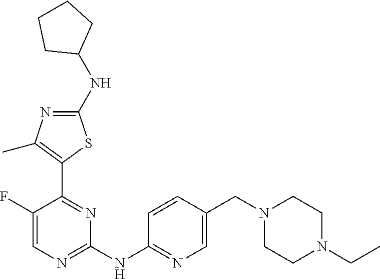 [SMILES: CCN1CCN(Cc2ccc(Nc3ncc(F)c(-c4sc(NC5CCCC5)nc4C)n3)nc2)CC1] | N-cyclopentyl-5-(2-((5-((4-ethylpiperazin-1- yl)methyl)pyridin-2-yl)amino)-5-fluoropyrimidin-4- yl)-4-methylthiazol-2-amine | 496.7 |
| 2 | 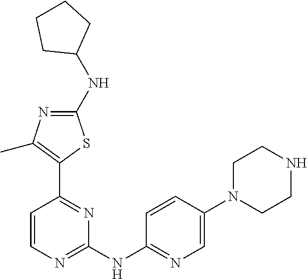 [SMILES: Cc1nc(NC2CCCC2)sc1-c1ccnc(Nc2ccc(N3CCNCC3)cn2)n1] | N-cyclopentyl-4-methyl-5-(2-((5-(piperazin-1- yl)pyridin-2-yl)amino)pyrimidin-4-yl)thiazol-2- amine | 436.2 |
| 3 | 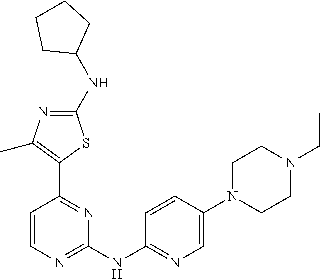 [SMILES: CCN1CCN(c2ccc(Nc3nccc(-c4sc(NC5CCCC5)nc4C)n3)nc2)CC1] | N-cyclopentyl-5-(2-((5-(4-ethylpiperazin-1- yl)pyridin-2-yl)amino)pyrimidin-4-yl)-4- methylthiazol-2-amine | 464.2 |
| 4 | 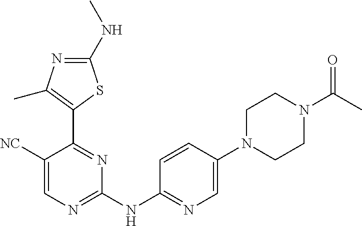 [SMILES: CNc1nc(C)c(-c2nc(Nc3ccc(N4CCN(C(C)=O)CC4)cn3)ncc2C#N)s1] | 2-((5-(4-acetylpiperazin-1-yl)pyridin-2-yl)amino)-4- (4-methyl-2-(methylamino)thiazol-5-yl)pyrimidine- 5-carbonitrile | 449.2 |
| 5 | 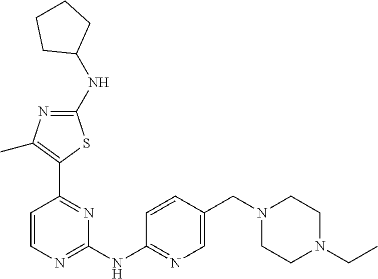 [SMILES: CCN1CCN(Cc2ccc(Nc3nccc(-c4sc(NC5CCCC5)nc4C)n3)nc2)CC1] | N-cyclopentyl-5-(2-((5-((4-ethylpiperazin-1- yl)methyl)pyridin-2-yl)amino)pyrimidin-4-yl)-4- methylthiazol-2-amine | 478.3 |
| 6 | 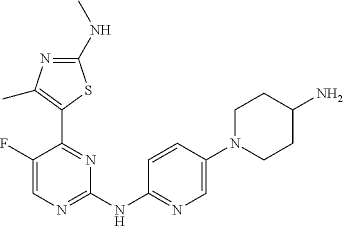 [SMILES: CNc1nc(C)c(-c2nc(Nc3ccc(N4CCC(N)CC4)cn3)ncc2F)s1] | 5-(2-((5-(4-aminopiperidin-1-yl)pyridin-2-yl)amino)- 5-fluoropyrimidin-4-yl)-N,4-dimethylthiazol-2-amine | 414.2 |
| 7 | 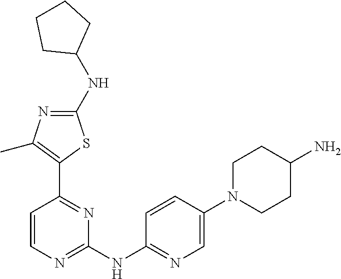 [SMILES: Cc1nc(NC2CCCC2)sc1-c1ccnc(Nc2ccc(N3CCC(N)CC3)cn2)n1] | 5-(2-((5-(4-aminopiperidin-1-yl)pyridin-2- yl)amino)pyrimidin-4-yl)-N-cyclopentyl-4- methylthiazol-2-amine | 450.2 |
| 8 | 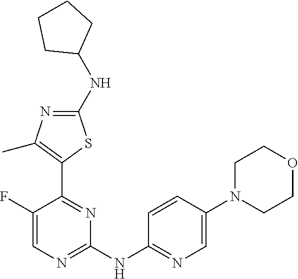 [SMILES: Cc1nc(NC2CCCC2)sc1-c1nc(Nc2ccc(N3CCOCC3)cn2)ncc1F] | N-cyclopentyl-5-(5-fluoro-2-((5-morpholinopyridin- 2-yl)amino)pyrimidin-4-yl)-4-methylthiazol-2-amine | 455.2 |
| 9 | 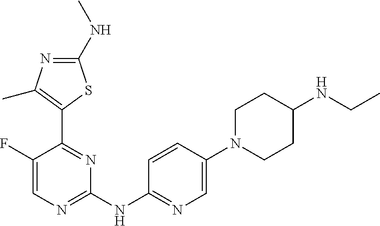 [SMILES: CCNC1CCN(c2ccc(Nc3ncc(F)c(-c4sc(NC)nc4C)n3)nc2)CC1] | 5-(2-((5-(4-(ethylamino)piperidin-1-yl)pyridin-2- yl)amino)-5-fluoropyrimidin-4-yl)-N,4- dimethylthiazol-2-amine | 442.2 |
| 10 | 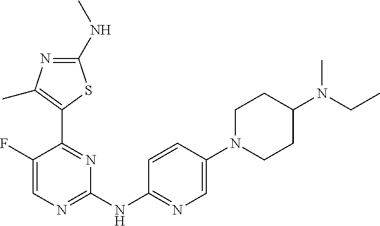 [SMILES: CCN(C)C1CCN(c2ccc(Nc3ncc(F)c(-c4sc(NC)nc4C)n3)nc2)CC1] | 5-(2-((5-(4-(ethyl(methyl)amino)piperidin-1- yl)pyridin-2-yl)amino)-5-fluoropyrimidin-4-yl)-N,4- dimethylthiazol-2-amine | 456.2 |
| 11 | 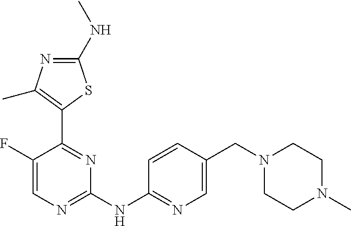 | 5-(5-fluoro-2-((5-((4-methylpiperazin-1- yl)methyl)pyridin-2-yl)amino)pyrimidin-4-yl)-N,4- dimethylthiazol-2-amine | 428.2 |
| 12 | 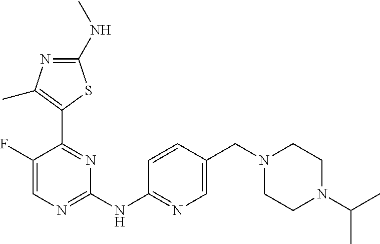 [SMILES: CNc1nc(C)c(-c2nc(Nc3ccc(CN4CCN(C(C)C)CC4)cn3)ncc2F)s1] | 5-(5-fluoro-2-((5-((4-isopropylpiperazin-1- yl)methyl)pyridin-2-yl)amino)pyrimidin-4-yl)-N,4- dimethylthiazol-2-amine | 456.2 |
| 13 | 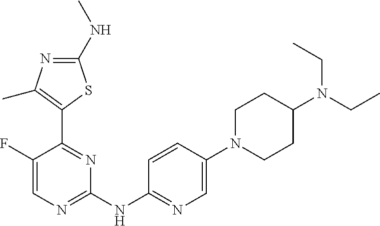 [SMILES: CCN(CC)C1CCN(c2ccc(Nc3ncc(F)c(-c4sc(NC)nc4C)n3)nc2)CC1] | 5-(2-((5-(4-(diethylamino)piperidin-1-yl)pyridin-2- yl)amino)-5-fluoropyrimidin-4-yl)-N,4- dimethylthiazol-2-amine | 470.2 |

The other immunotherapeutic agent may be selected from, for example, agents that are known to those skilled in the art as being capable of promoting an immunoreaction against cancer cells. For example, the other immunotherapeutic agent may be selected from immune checkpoint inhibitors such as any agent (which may be, for example, a protein, peptide, antibody or antibody fragment or a combination thereof) which is capable of inhibiting interactions between one or more immune checkpoint receptor and their ligand(s) (eg by blocking). As will be appreciated by those skilled in the art, among the immune checkpoint inhibitors suitable for use in the method of the first aspect are agents which are capable of inhibiting PD-1 (eg an anti-PD-i blocking antibody such as pembrolizumab, lambrolizumab, cemiplimab, spartalizumab and nivolumab; or an anti-PD-Li antibody such as atezolizumab, avelumab and durvalumab, or an anti-PD-L2 antibody), PD-L1, PD-L2, CTLA-4 (eg an anti-CTLA-4 antibody such as ipilimumab), B and T lymphocyte attenuator (BTLA; CD272) (eg an anti-BTLA antibody), the T cell immunoglobulin and mucin domain-3 (Tim-3; CD366) (eg an anti-Tim-3 antibody) or so-called intracellular checkpoints such as E3 ubiquitin-protein ligase (CBL-B) and CISH (cytokine-inducible SH2-containing protein).

It is to be appreciated that in the method of the first aspect, the method may comprise co-administering one or more compound of formula I (or a pharmaceutically acceptable salt, solvate or prodrug thereof) and one or more other immunotherapeutic agent.

In some embodiments, the method of the first aspect may further comprise administering one or more additional compound(s) with anti-proliferative activity, such as a compound(s) of one or more of the following categories:
Anti-neoplastic drugs and combinations thereof, as used in medical oncology, such as alkylating agents (eg cis-platin, oxaliplatin, carboplatin, cyclophosphamide, nitrogen mustard, melphalan, chlorambucil, busulphan, temozolamide and nitrosoureas); antimetabolites (eg gemcitabine and antifolates such as fluoropyrimidines like 5-fluorouracil and tegafur, raltitrexed, methotrexate, cytosine arabinoside, fludarabine and hydroxyurea); antitumour antibiotics (eg anthracyclines such as adriamycin, bleomycin, doxorubicin, daunomycin, epirubicin, idarubicin, mitomycin-C, dactinomycin and mithramycin); antimitotic agents (eg vinca alkaloids such as vincristine, vinblastine, vindesine and vinorelbine and taxoids including taxol and taxotere and polokinase inhibitors); and topoisomerase inhibitors (eg epipodophyllotoxins such as etoposide and teniposide, amsacrine, topotecan and camptothecin);
cytostatic agents such as antioestrogens (eg tamoxifen, fulvestrant, toremifene, raloxifene, droloxifene and iodoxyfene), antiandrogens (eg bicalutamide, flutamide, nilutamide and cyproterone acetate), LHRH antagonists or LHRH agonists (eg goserelin, leuprorelin and buserelin), progestogens (eg megestrol acetate), aromatase inhibitors (eg as anastrozole, letrozole, vorazole and exemestane) and inhibitors of 5α-reductase such as finasteride;
anti-invasion agents (eg c-Src kinase family inhibitors such as 4-(6-chloro-2,3-methylenedioxyanilino)-7-[2-(4-methylpiperazin-1-yl)ethoxy]-5-tetrahydropyran-4-yloxyquinazoline (AZD0530; International Patent Publication No WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib) and bosutinib (SKI-606)), and metalloproteinase inhibitors including marimastat, inhibitors of urokinase plasminogen activator receptor function or antibodies to heparanase;
inhibitors of growth factor function (eg growth factor antibodies and growth factor receptor antibodies such as the anti-erbB2 antibody trastuzumab (Herceptin™), the anti-EGFR antibody panitumumab, the anti-erbB1 antibody cetuximab (Erbitux, C225) and any growth factor or growth factor receptor antibodies disclosed by Stern et al., Crit Rev Oncol Hematol 54:11-29, 2005). Such inhibitors also include tyrosine kinase inhibitors such as inhibitors of the epidermal growth factor family (eg EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (eg Ras/Raf signalling inhibitors such as farnesyl transferase inhibitors including sorafenib (BAY 43-9006), tipifarnib (RI15777) and lonafarnib (SCH66336); and rapamycin (mTOR) inhibitors such as everolimus, sirolimus and temsirolimus), inhibitors of cell signalling through MEK and/or AKT kinases (eg selumetinib, and trametinib), c-kit inhibitors, abl kinase inhibitors, phosphoinositide-3-kinase (PI3K) inhibitors (eg alpelisib and duvelisib), Plt3 kinase inhibitors, CSF-1R kinase inhibitors, IGF receptor (insulin-like growth factor) kinase inhibitors; inhibitors of PARP (eg olaparib and rucaparib); aurora kinase inhibitors (eg AZD1152, PH739358, VX-680, MLN8054, R763, MP235, MP529, VX-528 and AX39459) and cyclin dependent kinase inhibitors such as CDK2 and/or CDK9 inhibitors;
antiangiogenic agents such as those which inhibit the effects of vascular endothelial growth factor (eg the anti-vascular endothelial cell growth factor antibody bevacizumab (Avastin™) and VEGF receptor tyrosine kinase inhibitors such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within International Patent Publication No WO 00/47212), compounds such as those disclosed in International Patent Publication Nos WO97/22596, WO 97/30035, WO 97/32856 and WO 98/13354, and compounds that work by other mechanisms (eg linomide, inhibitors of integrin avB3 function and angiostatin);
anti-proliferative/antineoplastic drugs and combinations thereof, as used in medical oncology, such as alkylating agents (eg carmustine, procarbazine, lomustine, vincristine, and TMZ); antimetabolites (eg gemcitabine and antifolates such as fluoropyrimidines like 5-fluorouracil and tegafur, raltitrexed, methotrexate, cytosine arabinoside, fludarabine and hydroxyurea); antitumour antibiotics (eg anthracyclines such as adriamycin, bleomycin, doxorubicin, daunomycin, epirubicin, idarubicin, mitomycin-C, dactinomycin and mithramycin); antimitotic agents (eg vinca alkaloids such as vincristine, vinblastine, vindesine and vinorelbine and taxoids including taxol and taxotere and polokinase inhibitors); and topoisomerase inhibitors (eg epipodophyllotoxins such as etoposide and teniposide, amsacrine, topotecan and camptothecin);
cytostatic agents such as antioestrogens (eg tamoxifen, fulvestrant, toremifene, raloxifene, droloxifene and iodoxyfene), antiandrogens (eg bicalutamide, flutamide, nilutamide and cyproterone acetate), LHRH antagonists or LHRH agonists (eg goserelin, leuprorelin and buserelin), progestogens (eg megestrol acetate), aromatase inhibitors (eg as anastrozole, letrozole, vorazole and exemestane) and inhibitors of 5α-reductase such as finasteride;
vascular damaging agents such as Combretastatin A4 and compounds disclosed in International Patent Publication Nos WO 99/02166, WO 00/40529, WO 00/41669, WO 01/92224, WO 02/04434 and WO 02/08213;
an endothelin receptor antagonist such as zibotentan (ZD4054) or atrasentan; and
antisense therapies such as those which are directed to the targets listed above, such as ISIS 2503, an anti-ras antisense.

The method of the first aspect will typically be applied to the treatment of cancer or another proliferative cell disease or condition in a human subject. However, the subject may also be selected from, for example, livestock animals (eg cows, horses, pigs, sheep and goats), companion animals (eg dogs and cats) and exotic animals (eg non-human primates, tigers, elephants etc).

Cancers and other proliferative cell diseases and conditions that may be treated in accordance with the method of the first aspect include biliary tract cancer, brain cancer and other cancers of the central nervous system (CNS) (including glioblastomas and medulloblastomas), neuroblastomas, breast cancer, cervical cancer, ovarian cancer (including those arising from epithelial cells, stromal cells, germ cells, and mesenchymal cells), choriocarcinoma, colorectal cancer, endometrial cancer, liver cancer, lung cancer, oesophageal cancer, gastric cancer, haematological neoplasms (including acute lymphocytic leukemia (ALL), chronic lymphocytic leukemia (CLL) and chronic myelogenous leukemia (CML), and acute myeloid leukaemia (AML), multiple myeloma, AIDS-associated leukemias and adult T-cell leukemia lymphoma, lymphomas (including Non-Hodgkin's lymphoma, Hodgkin's disease and lymphocytic lymphomas)), intraepithelial neoplasms (including Bowen's disease and Paget's disease), oral cancer (including squamous cell carcinoma), pancreatic cancer, prostate cancer, sarcomas (including leiomyosarcoma, rhabdomyosarcoma, liposarcoma, fibrosarcoma, and osteosarcoma), skin cancer (including melanoma, Kaposi's sarcoma, basocellular cancer, and squamous cell cancer), testicular cancer (including germinal tumours such as seminoma, non-seminoma teratomas, and choriocarcinomas), stromal tumours, germ cell tumours, thyroid cancer (including thyroid adenocarcinoma and medullar carcinoma), and renal cancer (including adenocarcinoma and Wilms' tumour).

In some embodiments, the cancers and other proliferative cell diseases and conditions treated in accordance with the method of the first aspect are characterised by over-expression of CDK4 and/or cyclin D including, for example, lung cancer (Wu et al., J Transl Med 9:38 (2011)), breast cancer (An et al., Am J Pathol 154(1):113-118 (1999)), brain cancers and other cancers of the CNS (eg glioblastomas) and colorectal cancer (Ikeda et al., Jap J Clin Med 54(4):1054-1059 (1996)). CDK4 and/or cyclin D over-expression may be determined by, for example, assessing the amount of mRNA encoding CDK4 and/or cyclin D in a suitable sample using any of the techniques well known to those skilled in the art (eg quantitative amplification techniques such as qPCR).

In some embodiments, the cancers and other proliferative cell diseases and conditions treated in accordance with the method of the first aspect are characterised by over-expression of CDK6 and/or cyclin D including, for example, T-cell acute lymphoblastic leukemia (ALL), brain cancers and other cancers of the CNS (eg medullablastoma) and colorectal cancer and (reviewed in Tadesse et al., Cell Cycle 14(20):3220-30 (2015)). CDK6 and/or cyclin D over-expression may be determined by, for example, assessing the amount of mRNA encoding CDK6 and/or cyclin D in a suitable sample using any of the techniques well known to those skilled in the art (eg quantitative amplification techniques such as qPCR).

As will be appreciated by those skilled in the art, the step of co-administering the compound of formula I and the other immunotherapeutic agent may involve administering the compound and agent simultaneously to the subject or otherwise sequentially in any order (eg within seconds or minutes (eg 10, 60 or 90 minutes) or even hours (eg within 2 to 48 hours)). As such, the compound of formula I and the other immunotherapeutic agent can be administered in, for example, the same pharmaceutical composition (ie for simultaneous administration) or in separate pharmaceutical compositions.

In a second aspect, the present disclosure provides a pharmaceutical composition for treating a proliferative disease or condition in a subject, comprising a compound of formula I as defined in the first aspect (or a pharmaceutically acceptable salt, solvate or prodrug thereof) and an immunotherapeutic agent, optionally in combination with a pharmaceutically acceptable carrier, diluent and/or excipient.

The compound of formula I and the other immunotherapeutic agent may be formulated into a pharmaceutical composition in therapeutically effective amounts (which may be lesser amounts than might be used for treatments where the compounds are used alone) with a pharmaceutically acceptable carrier, diluent and/or excipient. Examples of suitable carriers and diluents are well known to those skilled in the art, and are described in, for example, Remington's Pharmaceutical Sciences, Mack Publishing Co., Easton, PA 1995. Examples of suitable excipients for the various different forms of pharmaceutical compositions described herein may be found in the Handbook of Pharmaceutical Excipients, 2^{nd} Edition, (1994), Edited by A Wade and PJ Weller. Examples of suitable carriers include lactose, starch, glucose, methyl cellulose, magnesium stearate, mannitol, sorbitol and the like. Examples of suitable diluents include ethanol, glycerol and water. The choice of carrier, diluent and/or excipient may be made with regard to the intended route of administration and standard pharmaceutical practice.

A pharmaceutical composition comprising a compound of formula I and the other immunotherapeutic agent may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, dyes and even flavouring agents may be provided in the pharmaceutical composition. Examples of preservatives include sodium benzoate, sorbic acid and esters of p-hydroxybenzoic acid. Anti-oxidants and suspending agents may be also used.

A pharmaceutical composition comprising a compound of formula I and an immunotherapeutic agent may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration. For oral administration, particular use may be made of compressed tablets, pills, tablets, gellules, drops, and capsules. For other forms of administration, a pharmaceutical composition may comprise solutions or emulsions which may be injected intravenously, intraarterially, intrathecally, subcutaneously, intradermally, intraperitoneally or intramuscularly, and which are prepared from sterile or sterilisable solutions. A pharmaceutical composition comprising a compound of formula I and an immunotherapeutic agent may also be in form of suppositories, pessaries, suspensions, emulsions, lotions, ointments, creams, gels, sprays, solutions or dusting powders. A pharmaceutical composition may be formulated in unit dosage form (ie in the form of discrete portions containing a unit dose, or a multiple or sub-unit of a unit dose).

The compound of formula I and/or the immunotherapeutic agent may be provided as a pharmaceutically acceptable salt including, for example, suitable acid addition or base salts thereof. A review of suitable pharmaceutical salts may be found in Berge et al., J Pharm Sci 66:1-19 (1977). Salts are formed, for example with strong inorganic acids such as mineral acids (eg sulfuric acid, phosphoric acid or hydrohalic acids), with strong organic carboxylic acids, such as alkanecarboxylic acids of 1 to 4 carbon atoms which are unsubstituted or substituted (eg by halogen), such as acetic acid, with saturated or unsaturated dicarboxylic acids (eg oxalic, malonic, succinic, maleic, fumaric, phthalic or tetraphthalic acid), with hydroxycarboxylic acids (eg ascorbic, glycolic, lactic, malic, tartaric or citric acid), with amino acids (eg aspartic or glutamic acid), with benzoic acid, or with organic sulfonic acids (eg (C₁-C₄)-alkyl- or aryl-sulfonic acids which are unsubstituted or substituted by, for example, halogen) such as methane- or p-toluene sulfonic acid). In addition, the compound of formula I and/or the other immunotherapeutic agent may be provided in their various crystalline forms, polymorphic forms and (an)hydrous forms. In this regard, it will be well known to those skilled in the art that chemical compounds may be isolated in any of such forms by slightly varying the method of purification and or isolation from the solvents used in the synthetic preparation of such compounds.

In a third aspect, the present disclosure provides the use of a compound of formula I as defined in the first aspect (or a pharmaceutically acceptable salt, solvate or prodrug thereof) and an immunotherapeutic agent for treating a proliferative disease or condition in a subject.

In a fourth aspect, the present disclosure provides the use of a compound of formula I as defined in the first aspect (or a pharmaceutically acceptable salt, solvate or prodrug thereof) and an immunotherapeutic agent in the manufacture of a medicament for treating a proliferative disease or condition in a subject.

In a fifth aspect, the present disclosure provides a kit comprising first and second containers (eg vials), wherein the first container contains a compound of formula I as defined in the first aspect (or a pharmaceutically acceptable salt, solvate or prodrug thereof), and the second container contains an immunotherapeutic agent; optionally packaged with instructions for the use of the kit in the method of the first aspect.

Methods for synthesising compounds of formula I have been previously described (see, for example, International (PCT) patent publication No WO 2017/020065, the content of which is hereby incorporated by reference in its entirety). In some embodiments, compounds of formula I may be synthesised by adapting the following general synthetic scheme:

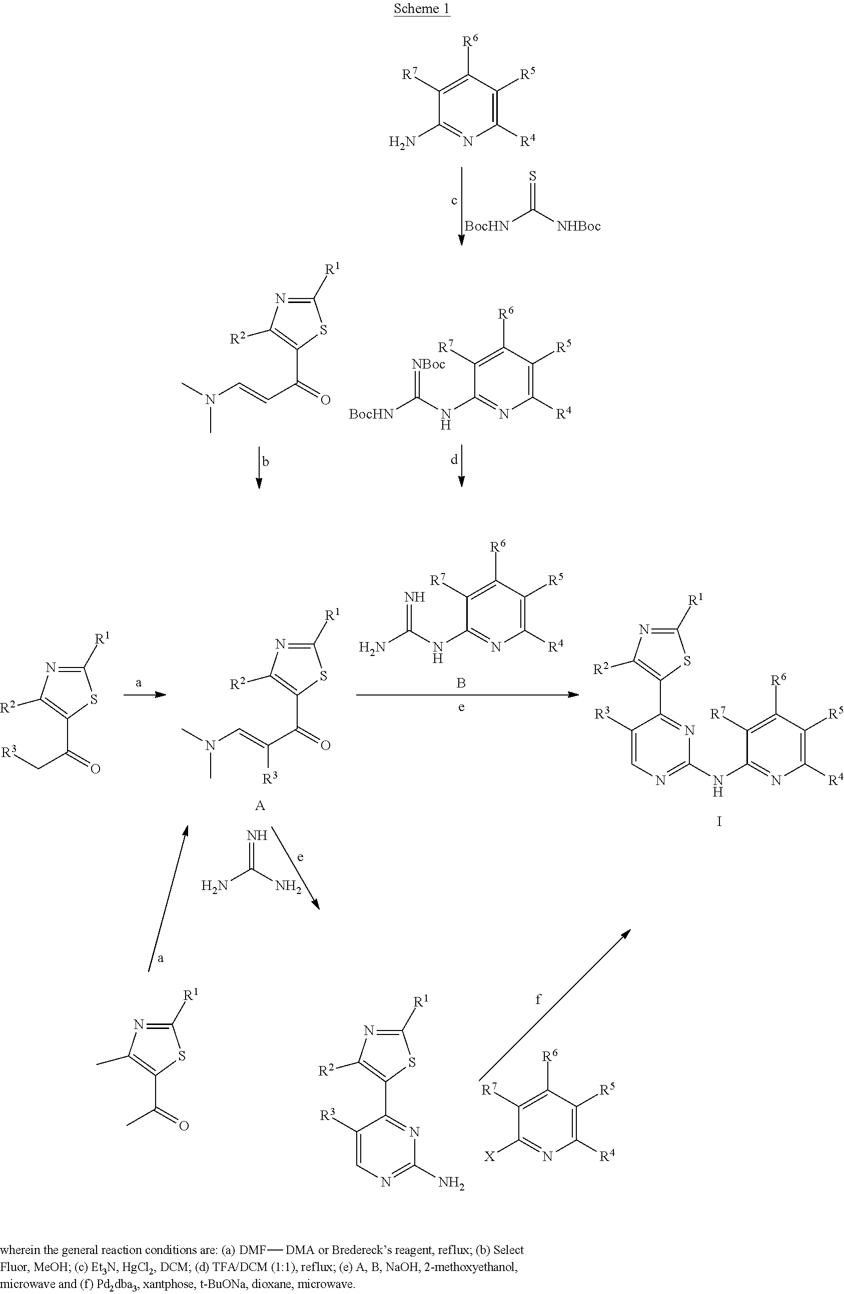

With regard to the description of the synthetic method of scheme 1 above, it will be understood by those skilled in the art that all proposed reaction conditions, including choice of solvent, reaction atmosphere, reaction temperature, duration of the experiment and workup procedures, can be readily selected. Moreover, it will be understood by those skilled in the art that the functionality present at various portions of the molecule must be compatible with the reagents and reaction conditions utilised.

Necessary starting materials may be obtained by standard procedures of organic chemistry. The preparation of such starting materials is described in conjunction with the following representative process variants and within the examples hereinafter. Alternatively, necessary starting materials may be obtainable by analogous procedures to those illustrated which are within the ordinary skill of those skilled in the art. Further, it will be appreciated that during the synthesis of the compounds, or during the synthesis of certain starting materials, it may be desirable to protect certain substituent groups to prevent their undesired reaction. Those skilled in the art will readily recognise when such protection is required, and how such protecting groups may be put in place, and later removed. Examples of protecting groups are described in, for example, Protective Groups in Organic Synthesis by Theodora Green (publisher: John Wiley & Sons). Protecting groups may be removed by any convenient method well known to those skilled in the art as appropriate for the removal of the protecting group in question, such methods being chosen so as to effect removal of the protecting group with the minimum disturbance of groups elsewhere in the molecule. Thus, if reactants include, for example, groups such as amino, carboxyl or hydroxyl, it may be desirable to protect the group in some of the reactions mentioned herein.

In addition, those skilled in the art will be able to select appropriate reaction conditions to use in the coupling reaction of the compound of formula A or formula B shown in scheme 1. However, typically, the reaction will be carried out in anhydrous conditions and in the presence of an inert atmosphere, such as argon or nitrogen. The reaction may also be carried out an elevated temperature, such as, for example, within the range of 80 to 180° C. for a suitable time period of, for example, 20 minutes to 48 hours. Suitably, the reaction is carried out under microwave heating, for example, at 80 to 180° C. for 20 minutes to 1.5 hour.

The resultant compound can be isolated and purified using techniques well known to those skilled in the art.

The methods and uses of the disclosure are hereinafter further described with reference to the following, non-limiting examples and accompanying figures.

### EXAMPLES

### Example 1 Synthesis of Representative Compounds

### General

¹H and ¹³C NMR spectra were recorded at 300 K on a Bruker AVANCE III 500 spectrometer (¹H at 500 MHz). ¹H NMR spectra were referenced to ¹H signals of residual non-deuterated solvents (or tetramethylsilane). High resolution mass spectra were recorded on an AB SCIEX TripleTOF® 5600 mass spectrometer, and ionisation of all samples was carried out using ESL The purity of compounds was determined by analytical HPLC, and was greater than 95%. Analytic HPLC was carried out on a Shimadzu Prominence UFLC (UltraFast Liquid Chromatograph) system with a CBM-20A communications bus module, a DGU-20A_{5R} degassing unit, an LC-20AD liquid chromatograph pump, an SIL-20A_{HT} autosampler, an SPD-M20A photo diode array detector, a CTO-20A column oven and a Phenomenex Kinetex 5u C18 100A 250 mm×4.60 mm column using Method A (gradient 5 to 95% MeOH containing 0.1% FA over 7 min, followed by 95% MeOH containing 0.1% FA over 13 min at a flow rate of 1 mL/min), Method B (gradient 5 to 95% MeCN containing 0.1% FA over 7 min followed by 95% MeCN containing 0.1% FA over 13 min, at a flow rate of 1 mL/min).

N-cyclopentyl-5-(2-((5-((4-ethylpiperazin-1-yl)methyl)pyridin-2-yl)amino)-5-fluoropyrimidin-4-yl)-4-methylthiazol-2-amine (1): To a solution of 5-(2-amino-5-fluoropyrimidin-4-yl)-N-cyclopentyl-4-methylthiazol-2-amine (200 mg, 0.68 mmol) in dioxane (3 mL) were added 1-((6-bromopyridin-3-yl)methyl)-4-ethylpiperazine (233. mg, 0.82 mmol), Pd2dba3 (31 mg, 0.034 mmol), xantphose (41 mg, 0.07 mmol) and t-BuONa (98 mg, 1.02 mmol) and heated under microwave irradiation at 150° C. for 1 h. The reaction mixture was cooled to room temperature and concentrated under reduced pressure. The residue was purified by chromatography (silica gel, DCM ramping to DCM:MeOH=93:7) to give 1 as an orange solid (100 mg, 29%). ¹H NMR (DMSO-d₆) δ 0.99 (t, 3H, J 7.0), 1.49-1.59 (m, 4H), 1.64-1.72 (m, 2H), 1.90-1.97 (m, 2H), 2.38 (s br, 10H), 2.48 (d, 3H, J 2.5), 3.42 (s, 2H), 3.95-3.98 (m, 1H), 7.64 (dd, 1H, J 8.5 & 2.0), 8.10 (d, 1H, J 8.5), 8.16 (d, 1H, J 2.0), 8.27 (d, 1H, J 7.0), 8.46 (d, 1H, J 3.5), 9.77 (s, 1H). HRMS (ESI): m/z 497.2601 [M+H]⁺.

N-cyclopentyl-4-methyl-5-(2-((5-(piperazin-1-yl) pyridin-2-yl) amino) pyrimidin-4-yl) thiazol-2-amine (2): To a mixture of crude 1-(5-(piperazin-1-yl)pyridin-2-yl)guanidine trifluoroacetate (441 mg, 2.00 mmol) and (E)-1-(2-(cyclopentylamino)-4-methylthiazol-5-yl)-3-(dimethylamino) prop-2-en-1-one (279 mg, 1.00 mmol) in 2-methoxy ethanol (3 mL) was added NaOH (80.0 mg, 2.00 mmol). The reaction mixture was heated at 180° C. under microwave irradiation for 1 h, cooled to room temperature and concentrated under reduced pressure. The residue was purified by chromatography (silica gel, DCM ramping to DCM:MeOH=92:8) and recrystallised with DCM and MeOH to give 2 as a dark yellow solid (70.0 mg, 16%). m.p. 210-213° C. ¹H NMR (DMSO-d₆) 1.49-1.68 (m, 7H), 1.89-1.94 (m, 2H), 2.46 (s, 3H), 2.85 (t, 4H, J 4.5), 3.02 (t, 4H, J 5.0), 3.98 (m, 1H), 6.90 (d, 1H, J 5.5), 7.36 (dd, 1H, J 9.0 & 3.0), 7.98 (d, 1H, J 3.0), 8.07 (d, 1H, J 9.0), 8.18 (d, 1H, J 7.0), 8.33 (d, 1H, J 5.5), 9.33 (s, 1H). HRMS (ESI): m/z 437.2222 [M+H]⁺.

N-cyclopentyl-5-(2-((5-(4-ethylpiperazin-1-yl)pyridin-2-yl)amino)pyrimidin-4-yl)-4-methylthiazol-2-amine (3): To a mixture of crude 1-(5-(4-ethylpiperazin-1-yl)pyridin-2-yl)guanidine trifluoroacetate (496 mg, 2.00 mmol) and (E)-1-(2-(cyclopentylamino)-4-methylthiazol-5-yl)-3-(dimethylamino) prop-2-en-1-one (279 mg, 1.00 mmol) in 2-methoxy ethanol (3 mL) was added NaOH (80.0 mg, 2.00 mmol). The reaction mixture was heated at 180° C. under microwave irradiation for 1 h, cooled to room temperature and concentrated under reduced pressure. The residue was purified by chromatography (silica gel, DCM ramping to DCM:MeOH=96:4) and recrystallised from MeOH to give 3 as a yellow solid (117 mg, 25%). ¹H NMR (CDCl₃) δ 1.14 (t, 3H, J 7.0), 1.56-1.76 (m, 6H), 2.06-2.12 (m, 2H), 2.49 (q, 2H, J 7.5), 2.54 (s, 3H), 2.64 (s, 3H), 3.19 (t, 4H, J 4.5), 3.14 (t, 4H, J 5.0), 3.86 (app s, 1H), 5.77 (s, 1H), 6.84 (d, 1H, J 5.0), 7.34 (dd, 1H, J 9.0 & 3.0), 7.94 (d, 1H, J 3.0), 7.94 (s, 1H), 8.01 (d, 1H, J 3.0), 8.26 (d, 1H, J 9.0), 8.33 (d, 1H, J 5.5). HRMS (ESI): m/z 465.2541 [M+H]⁺.

2-((5-(4-acetylpiperazin-1-yl)pyridin-2-yl)amino)-4-(4-methyl-2-(methylamino)thiazol-5-vl)pyrimidine-5-carbonitrile (4): To a solution of crude 1-(5-(4-acetylpiperazin-1-yl)pyridin-2-yl)guanidine trifluoroacetate (315 mg, 1.20 mmol) in 2-methoxyethanol (4 mL) were added tert-butyl (E)-(5-(2-cyano-3-(dimethylamino)acryloyl)-4-methylthiazol-2-yl)(methyl)carbamate (350 mg, 1.00 mmol) and NaOH (82.0 mg, 2.40 mmol). The reaction mixture was heated at 180° C. for 90 min under microwave irradiation, cooled down to room temperature, and then concentrated under reduced pressure. The residue was purified by chromatography (silica gel, DCM ramping to DCM:MeOH=90:10 with consecutive addition of 32% aqueous ammonia, up to 3%). The solid was washed with DCM and MeOH, then filtered to give 4 as a pale yellow solid (157 mg, 35%). ¹H NMR (DMSO-d) 8 2.04 (s, 3H), 2.40 (s, 3H), 2.87 (s, 3H), 3.10 (t, 2H, J 5.0), 3.16 (t, 2H, J 5.0), 3.58 (t, 4H, J 5.0), 7.46 (dd, 1H, J 9.5 & 3.0), 7.90 (d, 1H, J 9.0), 8.06 (d, 1H, J 3.0), 8.26 (q, 1H, J 3.0), 8.75 (s, 1H), 10.33 (s, 1H). HRMS (ESI): m/z 450.1844 [M+H]⁺.

N-cyclopentyl-5-(2-((5-((4-ethylpiperazin-1-yl)methyl)pyridin-2-yl)amino)pyrimidin-4-yl)-4-methylthiazol-2-amine (5): To a solution of 5-(2-aminopyrimidin-4-yl)-N-cyclopentyl-4-methylthiazol-2-amine (275 mg, 1.00 mmol) in dioxane (3 mL) were added 1-((6-bromopyridin-3-yl)methyl)-4-ethylpiperazine (341 mg, 1.2 mmol), Pd₂dba₃ (45.8 mg, 0.05 mmol), xantphose (58 mg, 0.1 mmol) and t-BuONa (144 mg, 1.5 mmol) and heated under microwave irradiation at 150° C. for 1 h. The reaction mixture was cooled to room temperature and concentrated under reduced pressure. The residue was purified by chromatography (silica gel, DCM ramping to DCM:MeOH:NH₄OH=9:1:0.3) and recrystallised with DCM and MeOH to give 5 as a white solid (200 mg, 42%). ¹H NMR (CDCl₃) δ 1.09 (t, 3H, J 7.0), 1.58-1.76 (m, 6H), 2.08-2.14 (m, 2H), 2.43 (q, 2H, J 7.0, CH₂CH₃), 2.55 (s br, 11H), 3.48 (s, 2H), 3.86-3.92 (m, 1H), 5.42 (d, 2H, J 7.0), 6.90 (d, 1H, J 5.5), 7.68 (dd, 1H, J 9.0 & 2.5), 7.89 (s, 1H), 8.19 (d, 1H, J 2.0), 8.35-8.38 (m, 2H). HRMS (ESI): m/z 479.2703 [M+H]⁺.

5-(2-((5-(4-aminopiperidin-1-yl)pyridin-2-yl)amino)-5-fluoropyrimidin-4-yl)-N,4-dimethylthiazol-2-amine (6): Compound 7 was obtained by reacting 1-(5-(4-aminopiperidin-1-yl)pyridin-2-yl)guanidine trifluoroacetate (702 mg, 3.00 mmol) and (469 mg, 2.00 mmol) and ((E)-3-(dimethylamino)-2-fluoro-1-(4-methyl-2-(methylamino)thiazol-5-yl)prop-2-en-1-one (243 mg, 1.00 mmol) to yield an orange solid (40.0 mg, 10%). ¹H NMR (DMSO-d6) δ 1.75-1.80 (m, 2H), 2.45 (d, 3H, J 2.0), 2.62 (t, 2H, J 6.0), 2.85 (t, 2H, J 5.5), 3.45 (t, 2H, J 5.0), 3.53 (t, 2H, J 6.0), 7.13 (dd, 1H, J 9.0 & 3.0), 7.78 (s, 1H), 7.79 (d, 1H, J 4.5), 8.08 (q, 1H, J 4.5), 8.37 (d, 1H, J 3.5), 9.21 (s, 1H). HRMS (ESI): m/z 415.1821 [M+H]⁺.

5-(2-((5-(4-aminopiperidin-1-yl)pyridin-2-yl)amino)pyrimidin-4-yl)-N-cyclopentyl-4-methylthiazol-2-amine (7): To a mixture of 1-(5-(4-aminopiperidin-1-yl)pyridin-2-yl)guanidine trifluoroacetate (702 mg, 3.00 mmol) and (E)-1-(2-(cyclopentylamino)-4-methylthiazol-5-yl)-3-(dimethylamino) prop-2-en-1-one (558 mg, 2.00 mmol) in 2-methoxy ethanol (5 mL) was added NaOH (160.0 mg, 4.00 mmol). The reaction mixture was heated at 180° C. under microwave irradiation for 2 h, cooled to room temperature and concentrated under reduced pressure. The residue was purified by chromatography to give 7 as a yellow solid (90 mg, 10%). ¹H NMR (CDCl₃) δ 1.50-1.77 (m, 10H), 1.95 (d, 2H, J 10.5), 2.07-2.13 (m, 2H), 2.54 (s, 3H), 2.75-2.85 (m, 3H), 3.53-3.56 (m, 2H), 3.85-3.91 (m, 1H), 5.43 (d, J 5.0, 1H), 6.84 (d, 1H, J 5.5), 7.34 (dd, 1H, J 9.0 & 3.0), 7.75 (s, 1H), 8.00 (d, 1H, J 3.0), 8.25 (d, 1H, J 9.0), 8.32 (d, 1H, J 5.5). HRMS (ESI): m/z 451.2415 [M+H]⁺.

N-cyclopentyl-5-(2-((5-morpholinopyridin-2-yl)amino)pyrimidin-4-yl)-4-methylthiazol-2-amine (8): To a mixture of 1-(5-morpholinopyridin-2-yl)guanidine trifluoroacetate (442 mg, 2.00 mmol) and (E)-1-(2-(cyclopentylamino)-4-methylthiazol-5-yl)-3-(dimethylamino)-2-fluoroprop-2-en-1-one (297 mg, 1.00 mmol) in 2-methoxy ethanol (3 mL) was added NaOH (80.0 mg, 2.00 mmol). The reaction mixture was heated at 180° C. under microwave irradiation for 1 h, cooled to room temperature and concentrated under reduced pressure. The residue was purified by chromatography to give 8 as a brown solid (120 mg, 26%). ¹H NMR (DMSO-d₆) δ 1.50-1.57 (m, 4H), 1.66-1.69 (m, 2H), 1.90-1.95 (m, 2H), 2.47 (d, 1H, J 2.5), 3.09 (t, 4H, J 5.0), 3.75 (t, 4H, J 5.0), 3.96 (m, 1H), 7.42 (dd, 1H, J 9.0 & 3.0), 7.96 (d, 1H, J 9.0), 7.98 (d, 1H, J 3.0), 8.24 (d, 1H, J 7.0), 8.41 (d, 1H, J 7.0), 9.52 (s, 1H). HRMS (ESI): m/z 456.1976 [M+H]⁺.

### Example 2 Pharmacological Activity of Representative Compounds Kinase Assays

Eurofins Pharma Discovery or Reaction Biology Corporation Kinase Profiler services were used to measure inhibition of CDKs and other kinases by radiometric assay (RIA). Inhibition of CDK4/cyclin D1, CDK6/cyclin D3 and CDK9/cyclin T1 were also determined using ADP Glo Kinase assays (Promega Corporation, Madison WI, United States of America). Briefly, the kinase reaction for CDK4/cyclin D1 and CDK6/cyclin D3 was performed with kinase reaction buffer (40 nM Tris base pH 7.5, 20 mM MgCl₂, 0.4 mM DTT), 0.1 mg/ml BSA and RB-CTF substrate (retinoblastoma protein1 C-terminal fraction). For CDK9/cyclin T1, the kinase reaction was performed with standard assay buffer and Kinase Dilution Buffer and RBER-IRStide substrate. Serial dilutions of 1:3 were prepared for test compounds for 10 concentrations (from 10 μM to 0.5 nM). The kinase reactions were started by addition of ATP, incubated for 40 min at 37° C. and then stopped by adding 10 μL of ADP Glo reagent. After incubation at room temperature (RT) in the dark for 40 min, 20 μL of kinase detection reagent was added per well and incubated for 40 min. Luminescence was measured using an EnVision Multilabel plate reader (PerkinElmer, Buckinghamshire, United Kingdom). Positive and negative controls were performed in the presence and absence of CDK kinases, respectively. Half-maximal inhibition (IC₅₀) values were calculated using a 4-parameter logistic non-linear regression model with Graphpad prism (Version 6.0). Apparent inhibition constants (Kᵢ) values were calculated from Kₘ (ATP) and IC₅₀ values for the respective kinases. The results of representative compounds are shown in Table 2. Compound 1 is the most potent CDK4/6 inhibitor and was selected for further assessment for immunotherapeutic potential.

TABLE 2
Inhibition of cyclin-dependant kinases

| | CDK inhibition Kᵢ (μM) or % remaining enzymatic activity at 10 μM | | | | | |
|---|---|---|---|---|---|---|
| Compound | CDK1/B | CDK2/A | CDK4/D1 | CDK6/D3 | CDK7/H | CDK9/T1 |
| 1 | >1 | 0.620 | 0.0002 | 0.003 | 0.630 | >1 |
| 2 | >1 | >1 | 0.001 | 0.034 | >1 | 0.220 |
| 3 | >1 | 0.355 | 0.002 | 0.011 | 0.780 | 0.141 |
| 4 | >1 | >1 | 0.004 | 0.030 | >1 | >1 |
| 5 | >1 | 0.416 | 0.006 | 0.009 | 0.630 | >1 |
| 6 | >1 | 0.206 | 0.003 | 0.032 | >1 | >1 |
| 7 | >1 | 0.278 | 0.001 | 0.008 | 0.282 | 0.508 |
| 8 | >1 | 0.104 | 0.006 | 0.020 | >1 | >1 |

### Cell Culture

Murine triple-negative breast cancer EMT6 tumour cells (ATCC® CRL-2755™) and colorectal cancer CT26.WT cells (ATCC® CRL-2638™) were cultured and expanded in RPMI-1640 medium (Roswell Park Memorial Institute Medium-1640) with L-glutamine and sodium bicarbonate (Sigma-Aldrich, Macquarie Park, NSW, Australia) supplemented with 10% heat-inactivated foetal bovine serum (FBS). Cells were cultured in an incubator at 5% CO₂ 37° C.

In Vivo Anti-Tumour Activity in Mice with a Breast Cancer Cell Syngeneic Allograft

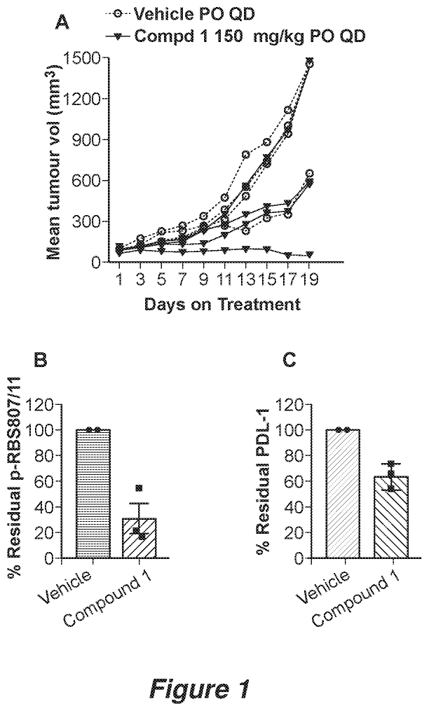

EMT6 cells were harvested and re-suspended in a 1:1 mixture of Matrigel™ (#354234; Corning Life Sciences, Corning, NY, United States of America) and serum-free RPMI-1640 medium, and thereafter injected sub-cutaneously (sc) (7×10⁵ cells in a volume of 100 L) into the right flank of each female BALB/c mouse. After the tumour volume reached ˜100 mm³, the mice were randomised into two groups (n=4/group) and given vehicle (1% CMC in water), or compound 1 (150 mg/kg) once daily by oral administration (QD PO). The treatment with compound12 resulted in tumour growth inhibition (% TGI) of 47% on the final day of tumour measurement (FIG. 1A). The level of phosphorylated RB (p-S807/11) was also found to be reduced in the EMT6 tumours collected two hours post-final dose, confirming CDK4/6 inhibition (FIG. 1B). A reduction in the immune checkpoint inhibitor, PDL-1, in the spleen samples of the mice isolated two hours post-final dose was also observed, indicating the immunotherapeutic potential of compound 1 (FIG. 1C). No significant weight loss or any overt toxicity was noticed in the compound 1-treated groups throughout the study.

In Vivo Anti-Tumour Activity in Mice with a Colorectal Cancer Cell Syngeneic Allograft

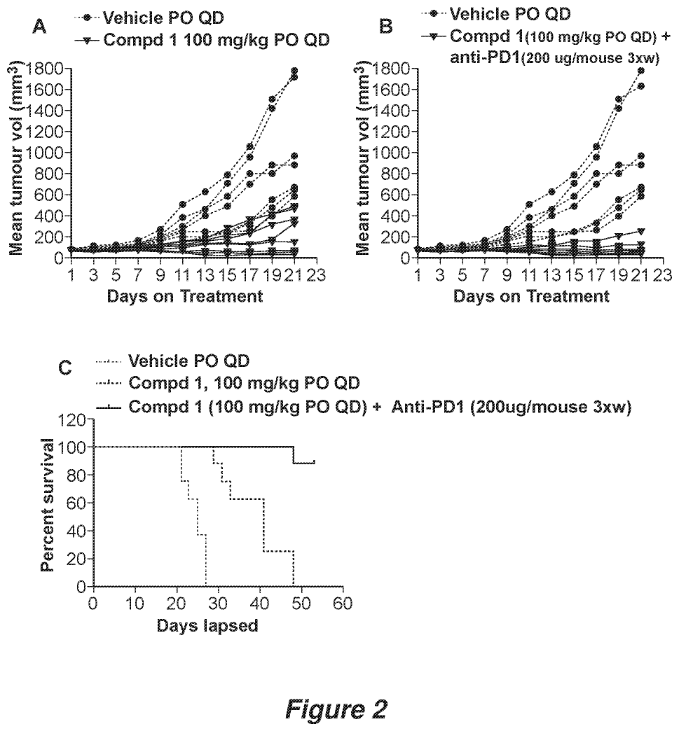

Similarly, mice bearing CT26.WT colorectal cancer cells were matched and randomised to vehicle or treatment groups (n=8/group). Female BALB/c mice with mean tumour volume ˜100 mm³ were given vehicle (distilled water QD PO), compound 1 (100 mg/kg QD PO), an anti-PD1 antibody (RMP1-14-derived mouse monoclonal antibody; Assay Matrix Pty Ltd, Ivanhoe North, VIC, Australia), 200 μg/mouse/week by intraperitoneal (ip) administration) or compound 1 plus anti-PD-1 antibody. Compound 1 achieved significant tumour growth inhibition (% TGI) of 75% when used alone, and 91% when used in the combination with the anti-PD-1 antibody, as measured on day 21 of dosing (FIG. 2A-B). The treatment with compound 1 was also found to significantly prolong the survival of the mice as a single agent (p<0.001) and when administered in combination with the anti-PD-1 antibody (p<0.001, FIG. 2C).

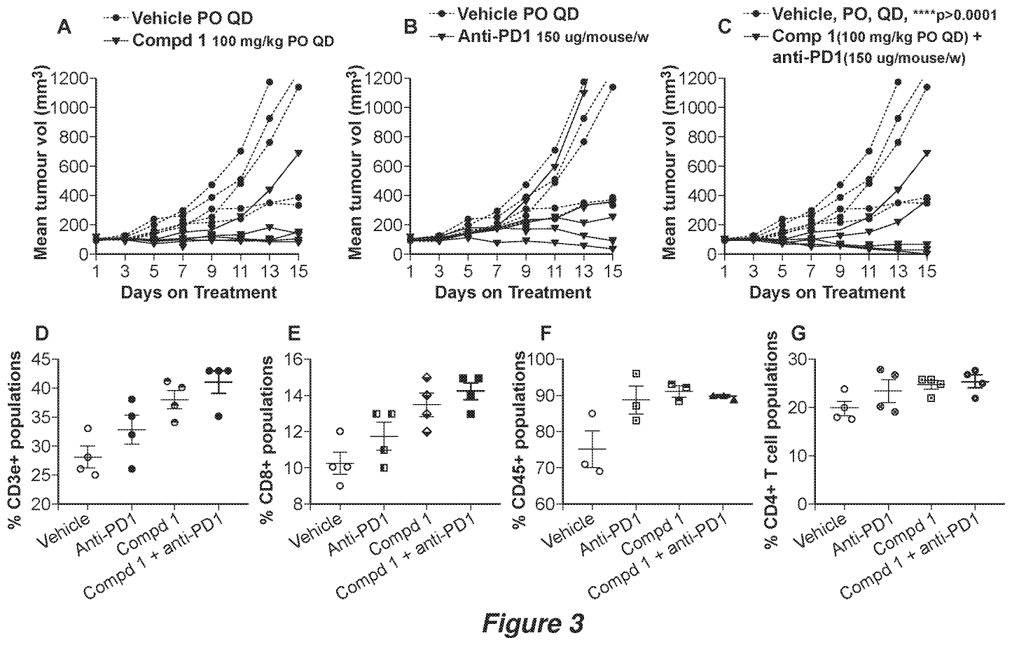

In another experiment, mice bearing CT26.WT colorectal cancer cells were treated with vehicle (distilled water QD PO), compound 1 (100 mg/kg QD PO), an anti-PD-1 antibody (150 g/mouse/week ip), or compound 1 plus the anti-PD-1 antibody. Compound 1 has caused TGI=77% when used alone and TGI=91% in the combination with anti-PD-1 antibody on day 15^{th} of dosing (FIGS. 3A and 3C). The combination approach was more effective compared with anti-PD-1 alone (p=0.03, FIG. 3B). The contribution of immunity toward the improved anti-cancer effects by compound 1 was demonstrated by the increased level of various immune markers such as CD3+, CD4+, CDK8+ and CD45+ T-cells (FIG. 3D-G).

### CONCLUSION

A representative compound of the group of compounds defined by Formula I, namely compound 1 (N-cyclopentyl-5-(2-((5-((4-ethylpiperazin-1-yl)methyl)pyridin-2-yl)amino)-5-fluoropyrimidin-4-yl)-4-methylthiazol-2-amine), was shown to inhibit the CDK4/6 kinase (known to be involved in the regulation of the cell cycle) and possess anti-tumour efficacy against mice with an intact immune system such as Balb/C mice bearing EMT6 breast cancer or CT26.WT colorectal cancer syngeneic allografts. Further, this representative compound was found to be capable of increasing the level of various immune markers, indicating that compounds of Formula I have excellent potential to act as an immunotherapeutic agent, especially when used in combination with another immunotherapeutic agent such as those used in checkpoint inhibitor therapy, for the treatment of proliferative cell diseases and conditions.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the present disclosure is not restricted in its use to the particular application described. Neither is the present disclosure restricted in its preferred embodiment with regard to the particular elements and/or features described or depicted herein. It will be also appreciated that the disclosure is not limited to the embodiment or embodiments disclosed, but is capable of numerous rearrangements, modifications and substitutions without departing from the scope of the disclosure as set forth and defined by the following claims.

1. A method of treating a proliferative disease or condition in a subject, comprising co-administering to the subject a compound of formula I shown below (or a pharmaceutically acceptable salt, solvate or prodrug thereof) with an immunotherapeutic agent: 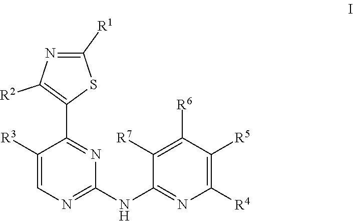
wherein:
R¹, R², R³, R⁴, R⁵, R⁶ and R⁷ are each independently selected from the group consisting of H, alkyl, aryl, aralkyl, halogen, NO₂, CN, CF₃, OH, O-alkyl, O-aryl, NH₂, NH-alkyl, NH-aryl, N-(alkyl)₂, N-(aryl)₂, N-(alkyl)(aryl), COOH, CONH₂, CONH-alkyl, CONH-aryl, SO₃H, SO₂-alkyl, SO₂-aryl, SO₂NH₂, CF₃, CO-alkyl, CO-aryl, wherein said alkyl, aryl and aralkyl groups may be optionally substituted with one or more groups selected from halogen, CN, OH, O-methyl, NH₂, COOH, CONH₂ and CF₃, and heterocyclic groups optionally substituted with one or more groups selected from alkyl, NH₂, NH-alkyl, N(alkyl)₂, COH and CO-alkyl;
but wherein the compound is not 5-(2-((5-(4-(dimethylamino)piperidin-1-yl)pyridin-2-yl)amino)-5-fluoropyrimidin-4-yl)-N,4-dimethylthiazol-2-amine.

2. The method of claim 1, wherein R¹ is H, C₁₋₆ alkyl, or an NH—C₁₋₆ alkyl.

3. The method of claim 2, wherein R¹ is NH-methyl or NH-cyclopentyl.

4. The method of claim 1, wherein R² is H, C₁₋₆ alkyl, CN or halogen.

5. The method of claim 1, wherein R³ is H, C₁₋₆ alkyl, CN or halogen.

6. The method of claim 1, wherein R⁴ and R⁷ are independently selected from H, O—C₁₋₆ alkyl and halogen.

7. The method of claim 1, wherein R⁵ and R⁶ are independently selected from H and saturated or unsaturated 5- or 6-membered heterocyclic groups comprising one or two N heteroatoms, optionally substituted with one or more groups selected from C₁₋₆ alkyl, NH₂, NH—C₁₋₆ alkyl, N(C₁₋₃ alkyl)₂, COH and CO—(C₁₋₃ alkyl).

8. The method of claim 7, wherein R⁵ and R⁶ are independently selected from H and saturated or unsaturated 5- or 6-membered heterocyclic groups comprising one or two N heteroatoms, optionally substituted with one or more groups selected from methyl, ethyl, C(CH₃)₂), NH₂, NH-methyl, NH-ethyl, N(CH₃)₂, N(CH₂CH₃)₂, N(CH₃)(CH₂CH₃), COH and COCH₃.

9. The method of claim 7, wherein R⁵ and R⁶ is/are independently selected from the following:

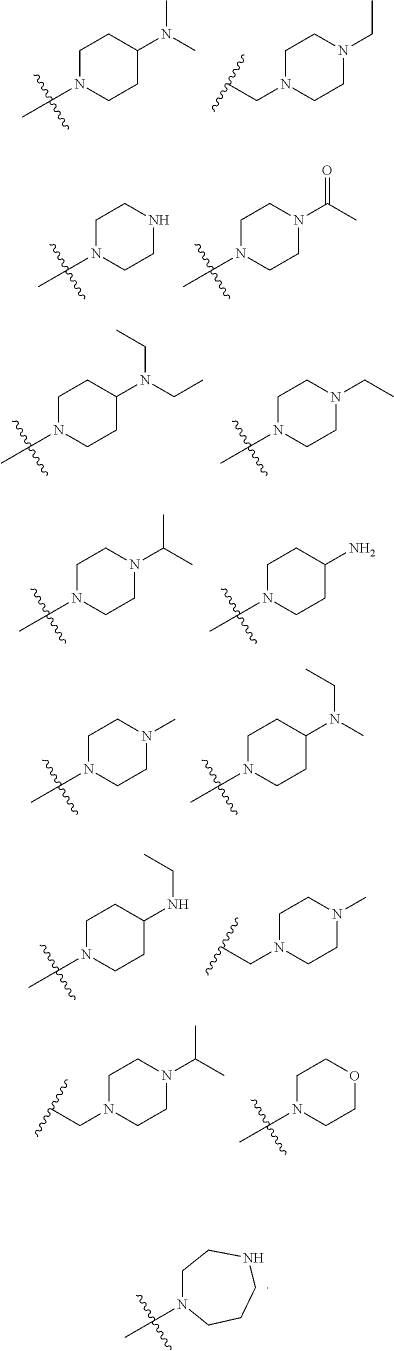

10. The method of claim 1, wherein R⁶ is H and R⁵ is selected from the following:

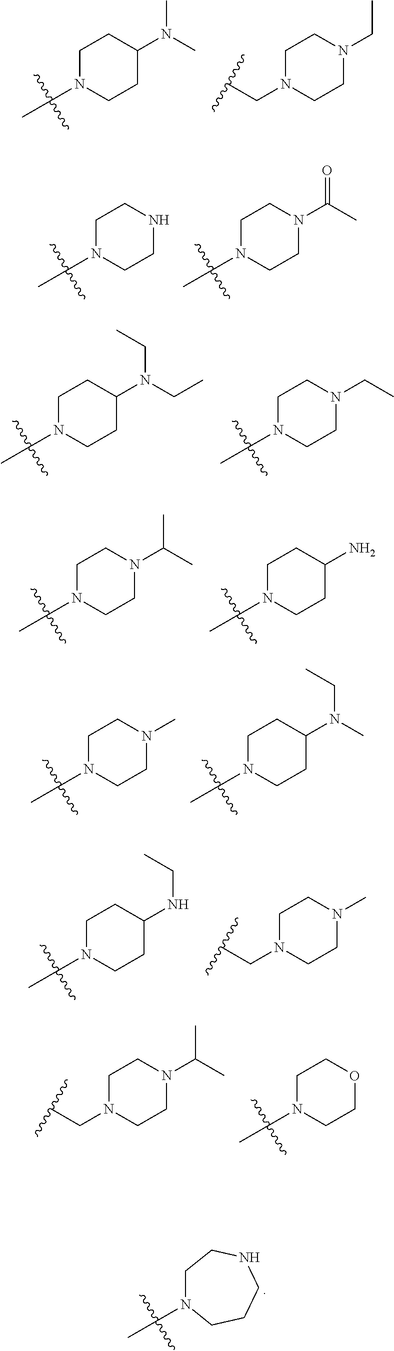

11. The method of claim 1, wherein the compound of formula I is:
N-cyclopentyl-5-(2-((5-((4-ethylpiperazin-1-yl)methyl)pyridin-2-yl)amino)-5-fluoropyrimidin-4-yl)-4-methylthiazol-2-amine;
N-cyclopentyl-4-methyl-5-(2-((5-(piperazin-1-yl)pyridin-2-yl)amino)pyrimidin-4-yl)thiazol-2-amine;
N-cyclopentyl-5-(2-((5-(4-ethylpiperazin-1-yl)pyridin-2-yl)amino)pyrimidin-4-yl)-4-methylthiazol-2-amine;
2-((5-(4-acetylpiperazin-1-yl)pyridin-2-yl)amino)-4-(4-methyl-2-(methylamino)thiazol-5-yl)pyrimidine-5-carbonitrile;
N-cyclopentyl-5-(2-((5-((4-ethylpiperazin-1-yl)methyl)pyridin-2-yl)amino)pyrimidin-4-yl)-4-methylthiazol-2-amine;
5-(2-((5-(4-aminopiperidin-1-yl)pyridin-2-yl)amino)-5-fluoropyrimidin-4-yl)-N,4-dimethylthiazol-2-amine;
5-(2-((5-(4-aminopiperidin-1-yl)pyridin-2-yl)amino)pyrimidin-4-yl)-N-cyclopentyl-4-methylthiazol-2-amine;
N-cyclopentyl-5-(5-fluoro-2-((5-morpholinopyridin-2-yl)amino)pyrimidin-4-yl)-4-methylthiazol-2-amine;
5-(2-((5-(4-(ethylamino)piperidin-1-yl)pyridin-2-yl)amino)-5-fluoropyrimidin-4-yl)-N,4-dimethylthiazol-2-amine;
5-(2-((5-(4-(ethyl(methyl)amino)piperidin-1-yl)pyridin-2-yl)amino)-5-fluoropyrimidin-4-yl)-N,4-dimethylthiazol-2-amine;
5-(5-fluoro-2-((5-((4-methylpiperazin-1-yl)methyl)pyridin-2-yl)amino)pyrimidin-4-yl)-N,4-dimethylthiazol-2-amine;
5-(5-fluoro-2-((5-((4-isopropylpiperazin-1-yl)methyl)pyridin-2-yl)amino)pyrimidin-4-yl)-N,4-dimethylthiazol-2-amine; or
5-(2-((5-(4-(diethylamino)piperidin-1-yl)pyridin-2-yl)amino)-5-fluoropyrimidin-4-yl)-N,4-dimethylthiazol-2-amine.

12. The method of claim 1, wherein the compound of formula I is:
N-cyclopentyl-5-(2-((5-((4-ethylpiperazin-1-yl)methyl)pyridin-2-yl)amino)-5-fluoropyrimidin-4-yl)-4-methylthiazol-2-amine;
N-cyclopentyl-4-methyl-5-(2-((5-(piperazin-1-yl)pyridin-2-yl)amino)pyrimidin-4-yl)thiazol-2-amine;
N-cyclopentyl-5-(2-((5-(4-ethylpiperazin-1-yl)pyridin-2-yl)amino)pyrimidin-4-yl)-4-methylthiazol-2-amine;
2-((5-(4-acetylpiperazin-1-yl)pyridin-2-yl)amino)-4-(4-methyl-2-(methylamino)thiazol-5-yl)pyrimidine-5-carbonitrile; or
N-cyclopentyl-5-(2-((5-((4-ethylpiperazin-1-yl)methyl)pyridin-2-yl)amino)pyrimidin-4-yl)-4-methylthiazol-2-amine.

13. The method of claim 1, wherein the compound of formula I is N-cyclopentyl-5-(2-((5-((4-ethylpiperazin-1-yl)methyl)pyridin-2-yl)amino)-5-fluoropyrimidin-4-yl)-4-methylthiazol-2-amine.

14. The method of claim 1, wherein the immunotherapeutic agent is selected from immune checkpoint inhibitors.

15. The method of claim 13, wherein the immunotherapeutic agent is selected from immune checkpoint inhibitors capable of inhibiting PD-1, PD-L1/2, CTLA-4, BTLA or Tim-3 and/or one or more of the ligands thereof.

16. The method of claim 15, wherein the immunotherapeutic agent is selected from pembrolizumab, lambrolizumab, cemiplimab, spartalizumab, nivolumab, atezolizumab, avelumab, durvalumab and ipilimumab.

17. The method of claim 15, wherein the immunotherapeutic agent is selected from immune checkpoint inhibitors capable of inhibiting PD-1 or a ligand thereof, namely PD-L1 or PD-L2.

18. The method of claim 1, wherein the proliferative disease or condition is selected from the group consisting of biliary tract cancer, brain cancer and other cancers of the central nervous system (CNS), neuroblastomas, breast cancer, cervical cancer, ovarian cancer, choriocarcinoma, colorectal cancer, endometrial cancer, liver cancer, lung cancer, oesophageal cancer, gastric cancer, haematological neoplasms, intraepithelial neoplasms, oral cancer, pancreatic cancer, prostate cancer, sarcomas, skin cancer, testicular cancer, stromal tumours, germ cell tumours, thyroid cancer, and renal cancer.

19. A pharmaceutical composition for treating a proliferative disease or condition in a subject, comprising a compound of formula I as defined in claim 1 (or a pharmaceutically acceptable salt, solvate or prodrug thereof) and an immunotherapeutic agent, optionally in combination with a pharmaceutically acceptable carrier, diluent and/or excipient.

20. The composition of claim 19, wherein the immunotherapeutic agent is selected from immune checkpoint inhibitors.

21. The composition of claim 20, wherein the immunotherapeutic agent is selected from immune checkpoint inhibitors capable of inhibiting PD-1, PD-L1/2, CTLA-4, BTLA or Tim-3 and/or one or more of the ligands thereof.

22. The composition of claim 21, wherein the immunotherapeutic agent is selected from pembrolizumab, lambrolizumab, cemiplimab, spartalizumab, nivolumab, atezolizumab, avelumab, durvalumab and ipilimumab.

23. The composition of claim 21, wherein the immunotherapeutic agent is selected from immune checkpoint inhibitors capable of inhibiting PD-1 or a ligand thereof, namely PD-L1 or PD-L2.

24-29. (canceled)

30. A kit comprising first and second containers, wherein the first container contains a compound of formula I as defined in claim 1 (or a pharmaceutically acceptable salt, solvate or prodrug thereof), and the second container contains an immunotherapeutic agent; optionally packaged with instructions for the use of the kit in the method of claim 1.

31-34. (canceled)
